(19) United States
(12) Patent Application Publication
(10) Pub. No.: US 2024/0239868 A1
(43) Pub. Date: Jul. 18, 2024

(54) POLYMERIC IMMUNOGLOBULIN RECEPTOR MUTANTS AND USES THEREOF

(71) Applicant: Shiying Cai, Cheshire, CT (US)

(72) Inventor: Shiying Cai, Cheshire, CT (US)

(73) Assignee: Shiying Cai, Cheshire, CT (US)

(21) Appl. No.: 18/407,775

(22) Filed: Jan. 9, 2024

Related U.S. Application Data
(60) Provisional application No. 63/438,761, filed on Jan. 12, 2023.

(51) Int. Cl. C07K 14/735; A61P 37/06

(52) U.S. Cl. CPC C07K 14/70535; A61P 37/06; A61K 38/00; C07K 2319/70

(57) ABSTRACT

Provided are polymeric immunoglobulin receptor mutants and uses thereof. Specifically, the present disclosure relates to a polymeric immunoglobulin receptor (pIgR) mutant for neutralizing an autoantibody against pIgR without interacting with a polymeric immunoglobulin, comprising an extracellular portion of wild-type pIgR with at least one amino acid mutation therein. The present disclosure also relates to an isolated nucleic acid encoding the amino acid sequence of the pIgR mutant, an expression vector comprising the isolated nucleic acid, a host cell comprising the expression vector, a pharmaceutical composition comprising the pIgR mutant and a method of treating an autoimmune disease.

### CROSS-REFERENCE TO RELATED APPLICATION

This application claims the benefit of U.S. Provisional Patent Application Ser. No. 63/438,761, filed Jan. 12, 2023, the contents of which are herein incorporated by reference in its entirety.

### STATEMENT REGARDING FEDERALLY SPONSORED RESEARCH OR DEVELOPMENT

(Not Applicable)

### REFERENCE TO ELECTRONIC SEQUENCE LISTING

This application contains a Sequence Listing which has been submitted electronically in .XML format and is hereby incorporated by reference in its entirety. Said. XML copy is created on Jan. 4, 2024 and named “EIC23310012P.xml”. The sequence listing contained in this. XML file is a part of the specification and hereby incorporated by reference herein in its entirety.

### TECHNICAL FIELD

The present disclosure relates to the biomedical field. In particular, the present disclosure relates to polymeric immunoglobulin receptor (pIgR) mutants, pharmaceutical compositions comprising the pIgR mutants, nucleic acids encoding the pIgR mutants, and uses of the pIgR mutants for the treatment of autoimmune diseases.

### BACKGROUND

Polymeric immunoglobulin receptor (pIgR) plays a critical role in innate immunity by facilitating the transcytosis of polymeric immunoglobulins, such as dIgA and pIgM from the basolateral side to the apical side of mucosal epithelial cells, including enterocytes, cholangiocytes, hepatocytes and bronchial epithelia. In these mucosal epithelia, pIgR is first presented as a membrane protein on the basolateral side where it binds to polymeric immunoglobulins through interacting with the J-chain that is covalently linked to the polymeric immunoglobulins as well as the polymeric immunoglobulin themselves. The complex then undergoes endocytosis and is translocated to the apical membrane through microtubule-endosome mechanism. Once reached the apical membrane, the extracellular portion of pIgR is cleaved and the complex is released to the mucus.

Recent studies indicate that pIgR is involved in the pathogenesis of certain autoimmune diseases such as primary biliary cholangitis (PBC), where it may act as a self-antigen. When the autoantibodies interact with pIgR on the basolateral membrane of mucosal epithelia, this immunocomplex triggers an auto-immunoresponse, leading to tissue injury. However, there is no therapeutic strategy for autoimmune diseases based on these studies have been reported yet.

PBC is an autoimmune liver disease that predominantly affects middle-aged women. The etiology of PBC remains unclear. Currently, there is no cure for this disease, although FDA-approved standard treatment is ursodeoxycholic acid that slows down the progression of the disease. However, there are more than 40% of patients not responding to this treatment. Obeticholic acid is also approved by FDA with restricted use in PBC patients, but adverse side effects have been observed. Therefore, new treatment is needed.

### SUMMARY OF THE INVENTION

The present disclosure relates in part to the finding that the pIgR plays a key role in the pathogenesis of the autoimmune diseases such as PBC as a self-antigen, because pIgR is highly expressed in the damaged small cholangiocytes, and autoantibodies against pIgR have been found in the serums of these patients, and enriched plasma cells were also found around the small bile ducts in these patient livers. Therefore, neutralizing the autoantibodies against the self-antigen pIgR can stop the autoimmunity and cure the autoimmune diseases.

In one aspect, the present disclosure provides a polymeric immunoglobulin receptor (pIgR) mutant for neutralizing an autoantibody against pIgR without interacting with a polymeric immunoglobulin, comprising an extracellular portion of wild-type pIgR with at least one amino acid mutation therein.

In some embodiments, the mutation is insertion, deletion and/or replacement of one or more amino acid residues in the amino acid sequence of the extracellular portion of wild-type pIgR.

In preferred embodiments, the extracellular portion of wild-type pIgR is the extracellular portion of wild-type human pIgR.

In some embodiments, the amino acid sequence of the extracellular portion of wild-type human pIgR is shown in SEQ ID NO: 2.

In some embodiments, the mutation occurs at one or more positions selected from a group consisting of positions 33-55, 114-117 and 486 in SEQ ID NO: 2.

In preferred embodiments, the mutation occurs at one or more positions selected from a group consisting of positions 48-50, 114-117 and 486 in SEQ ID NO: 2.

In further preferred embodiments, the pIgR mutant comprises one or more replacements of amino acid residues in SEQ ID NO: 2 selected from a group consisting of:
(i) ₄₈NRH to ₄₈TEL, or ₄₈NRH to ₄₈TAL;
(ii) ₁₁₄INSR to ₁₁₄ALSI, or ₁₁₄INSR to ₁₁₄ALST; and
(iii) C486S.

In some embodiments, the pIgR mutant further comprises at least one polypeptide tag.

In preferred embodiments, the polypeptide tag is selected from a group consisting of a 6×His tag, a human IgG Fc portion such as human IgG1 or human IgG2, and a carrier protein such as albumin.

In further preferred embodiments, the 6×His tag or the human IgG Fc portion is inserted at the carboxyl terminus of the pIgR mutant.

In further preferred embodiments, the carrier protein is conjugated with the pIgR mutant at position S637 in SEQ ID NO: 2.

In some embodiments, the amino acid sequence of the pIgR mutant comprises or consists of an amino acid sequence corresponding to positions 19-637 of any of SEQ ID NOs: 4-37.

In some embodiments, the amino acid sequence of the pIgR mutant is shown in any of SEQ ID NOs: 4-37.

In preferred embodiments, the amino acid sequence of the pIgR mutant comprises or consists of an amino acid sequence corresponding to positions 19-637 of any of SEQ ID NOs: 4-20, or has a sequence identity of at least 90%, such as 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% to an amino acid sequence corresponding to positions 19-637 of any of SEQ ID NOs: 4-20.

In preferred embodiments, the amino acid sequence of the pIgR mutant is shown in any of SEQ ID NOs: 4-20, or has a sequence identity of at least 90%, such as 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% to any of SEQ ID NOs: 4-20.

In some embodiments, the pIgR mutant does not interact with dIgA and/or pIgM.

In another aspect, the present disclosure provides an isolated nucleic acid encoding the amino acid sequence of the pIgR mutant described herein.

In yet another aspect, the present disclosure provides an expression vector comprising the isolated nucleic acid described herein.

In some embodiments, the expression vector further comprises a promoter that drives gene expression in mammalian cells, such as a CMV promoter.

In some embodiments, the expression vector is a plasmid vector.

In preferred embodiments, the plasmid vector is pcDNA3 or pcDNA3.1.

In some embodiments, the expression vector is a virus.

In preferred embodiments, the expression vector is an adenovirus, an adenovirus-associated vector, or a lentivirus.

In still another aspect, the present disclosure provides a host cell comprising the expression vector described herein.

In yet another aspect, the present disclosure provides a pharmaceutical composition comprising the pIgR mutant, the isolated nucleic acid, the expression vector or the host cell described herein, and optionally a pharmaceutically acceptable excipient, carrier or vehicle.

In still another aspect, the present disclosure provides a method of treating, inhibiting, reducing the severity of, slowing progression of and/or promoting prophylaxis of an autoimmune disease in a subject in need thereof comprising:

administering to the subject an effective amount of the pIgR mutant, or the pharmaceutical composition described herein.

In yet another aspect, the present disclosure provides a method of treating, inhibiting, reducing the severity of, slowing progression of and/or promoting prophylaxis of an autoimmune disease in a subject in need thereof comprising:
administering to the subject an effective amount of the isolated nucleic acid, the expression vector or the pharmaceutical composition described herein; and
expressing the pIgR mutant in vivo in the subject.

In some embodiments, the autoimmune disease is selected from a group of consisting of primary biliary cholangitis and autoimmune hepatitis.

In still another aspect, the present disclosure provides a method for preparation of the pIgR mutant described herein comprising:
culturing the host cell described herein under a condition suitable for expressing the pIgR mutant.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts the scheme of the working principle of the present disclosure, wherein 1: endogenous pIgR on the mucosal epithelia; 2: exogenously expressed or synthesized mutated pIgR proteins or peptides, i.e., pIgR mutants; 3: autoantibodies that interact with the self-antigen pIgR; 4: the mucosal epithelia, e.g. small bile duct cells; 5: immune cells, e.g. T-cell, NK cell, or macrophages; 6: polymeric immunoglobulins, e.g. dIgA and pIgM; 7: autoimmunoresponse damaged mucosal epithelia; 8: degraded autoantibodies after being neutralized/interacted with pIgR mutants.

FIG. 2 depicts the result of Example 2, demonstrating the specific site-directed mutations of human pIgR disable its interaction with dIgA and pIgM. Panel A: layout of the dot blot, wherein 1: wild-type; 2: V28A mutant; 3: ₄₈NRH to TAL and ₁₁₄INSR to ALSI mutant; 4: ₄₈NRH to TEL mutant; 5: ₄₈NRH to TAL mutant. Panel B: incubated with horseradish peroxidase (HRP) conjugated human dIgA. Panel C: incubated with HRP conjugated human pIgM.

FIG. 3 depicts the result of Example 3, demonstrating human pIgR mutants interact with the autoantibodies from PBC patients. Panel A: layout of the dot blot, wherein 1: BSA as a negative control; 2: wild-type; 3: ₄₈NRH to TAL mutant; 4: ₄₈NRH to TAL and C486S mutant; 5: ₄₈NRH to TAL, C486S and ₁₁₄INSR to ALSI mutant. Panel B: incubated with a rabbit polyclonal antibody from a commercial source. Panel C: incubated with 1:50 diluted serum from a PBC patient and detected with HRP conjugated anti-human IgG secondary antibody.

### DETAILED DESCRIPTION

All references cited herein are incorporated by reference in their entireties as though fully set forth. Unless defined otherwise, technical and scientific terms used herein have the same meaning as commonly understood by one of ordinary skill in the art to which this invention belongs. Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3rd ed., revised ed., J. Wiley & Sons (New York, NY 2006); Smith, March's Advanced Organic Chemistry Reactions, Mechanisms and Structure 7th ed., J. Wiley & Sons (New York, NY 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, N Y 2012), provide one skilled in the art with a general guide to many of the terms used in the present application.

One skilled in the art will recognize many methods and materials similar or equivalent to those described herein, which could be used in the practice of the invention. Other features and advantages of the invention will become apparent from the following detailed description, taken in conjunction with the accompanying drawings, which illustrate, by way of example, various features of embodiments of the invention. Indeed, the invention is in no way limited to the methods and materials described. For convenience, some terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to”, the term “having” should be interpreted as “having at least”, the term “includes” should be interpreted as “includes but is not limited to”, etc.).

Unless stated otherwise, the terms “a”, “an”, “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to some embodiments herein is intended merely to better illuminate the application and does not pose a limitation on the scope of the application otherwise claimed. The abbreviation, “e.g.” is derived from the Latin exempli gratia, and used herein to indicate a non-limiting example. Thus, the abbreviation “e.g.” is synonymous with the term “for example”. No language in the specification should be construed as indicating any non-claimed element essential to the practice of the application.

As used herein, the term “about” means plus or minus 10% of the numerical value of the number with which it is being used. Therefore, about 50% means in the range of 45%-55%.

As used herein, the term “optional” or “optionally” may be taken to mean that the subsequently described structure, event or circumstance may or may not occur, and that the description includes both instances where the event occurs and instances where it does not.

As used herein, the term “beneficial results” may include, but are in no way limited to, lessening or alleviating the severity of the disease condition, preventing the disease condition from worsening, preventing the disease condition from developing, lowering the chances of a patient developing the disease condition and prolonging a patient's life or life expectancy. Beneficial or desired clinical results include, but are not limited to, alleviation of one or more symptom(s), diminishment of extent of the deficit, stabilized (i.e., not worsening) state of the diseases progression, delay or slowing of the diseases, and amelioration or palliation of symptoms associated with the diseases. Treatment also includes a decrease in mortality or an increase in the lifespan of a subject as compared to one not receiving the treatment.

As used herein, the term “administering” and/or “administer” refer to any route for delivering a mutant, a pharmaceutical composition, an isolated nucleic acid or an expression vector to a patient. In one embodiment, the compositions described herein are administered enterically to the small intestine. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

As used herein, the term “effective amount” refers to the amount of a mutant, a pharmaceutical composition, an isolated nucleic acid or an expression vector to decrease at least one or more symptom of the disease or disorder, and relates to a sufficient amount of a mutant, a pharmaceutical composition, an isolated nucleic acid or an expression vector to provide the desired effect. The phrase “therapeutically effective amount” as used herein means a sufficient amount of a mutant, a pharmaceutical composition, an isolated nucleic acid or an expression vector to treat a disorder, at a reasonable benefit/risk ratio applicable to any medical treatment.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering the mutant, the pharmaceutical composition, the isolated nucleic acid or the expression vector described herein. Measured or measurable parameters include clinically detectable markers of disease, for example, elevated or depressed levels of a biological marker, as well as parameters related to a clinically accepted scale of symptoms or markers for the diseases. It will be understood, however, that the total daily usage of the mutant, the pharmaceutical composition, the isolated nucleic acid or the expression vector as disclosed herein will be decided by the attending physician within the scope of sound medical judgment. The exact amount required will vary depending on factors such as the type of disease being treated, gender, age, and weight of the subject.

As used herein, the term “subject” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. The term does not denote a particular age or sex. Thus, adult and newborn subjects, as well as fetuses, whether male or female, are intended to be included within the scope of this term.

As used herein, the term “treat/treatment/treating” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein, the term “amino acid” not only encompasses the 20 common amino acids in naturally synthesized proteins, but also includes any modified, unusual, or synthetic amino acid. One of ordinary skill in the art would be familiar with modified, unusual, or synthetic amino acids.

As used herein, the term “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The terms “polynucleotide sequence” and “nucleotide sequence” are also used interchangeably herein.

As used herein, the term “recombinant” as used herein, means that a protein is derived from a prokaryotic or eukaryotic expression system.

As used herein, the term “wild-type”, “native” or “endogenous” (used interchangeably), refers to the naturally-occurring polynucleotide sequence encoding a protein, or a portion thereof, or protein sequence, or portion thereof, respectively, as it normally exists in vivo.

As used herein, the term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent).

As used herein, the term “nucleic acid” will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A”, a guanine “G”, a thymine “T”, or a cytosine “C”) or RNA (e.g., an A, a G, an uracil “U” or a C). The term “nucleic acid” encompasses the terms “oligonucleotide” and “polynucleotide” each as a subgenus of the term “nucleic acid”. The term “oligonucleotide” refers to a molecule of between 3 and about 100 nucleobases in length. The term “polynucleotide” refers to at least one molecule of greater than about 100 nucleobases in length.

As used herein, the term “isolated” or “purified” polypeptide as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated.

Polymeric Immunoglobulin Receptor (pIgR) Mutants

Polymeric immunoglobulin receptor (pIgR) is a transmembrane protein with a signal peptide of 18AA in length. Its full-length in humans contains 764AA, with the matured form on the basolateral membrane as 746AA. It has 5 domains in its extracellular portion, with Domain 1 interacting with the J-chain that covalently links polymeric immunoglobulins, whereas the other domains facilitate/stabilize the interaction of pIgR and polymeric immunoglobulins. As a self-antigen, the extracellular portion of pIgR (about 620AA) is recognized by the autoantibodies. Typically, the amino acid sequence of wild-type human pIgR is shown in SEQ ID NO: 1, and its extracellular portion (also called secreted fragment) is shown in SEQ ID NO: 2.

The inventors' recent studies on PBC patients show that pIgR is highly expressed in the damaged small cholangiocytes, autoantibodies against pIgR have been found in the serum of these patients, and enriched plasma cells were also found around the small bile ducts in these patient livers. Therefore, the inventors conclude that the pIgR plays a key role in the pathogenesis of PBC as a self-antigen and targeting the autoantibodies against the pIgR may be a valuable approach to treat autoimmune diseases such as PBC.

The present disclosure provides a pIgR mutant comprising the extracellular portion of wild-type pIgR with at least one amino acid mutation therein that disables its interaction with polymeric immunoglobulins such as dIgA and pIgM, but has no or very limited effects in altering its property as an antigen. Therefore, the pIgR mutant can neutralize the autoantibodies against the pIgR to treat autoimmune diseases such as PBC.

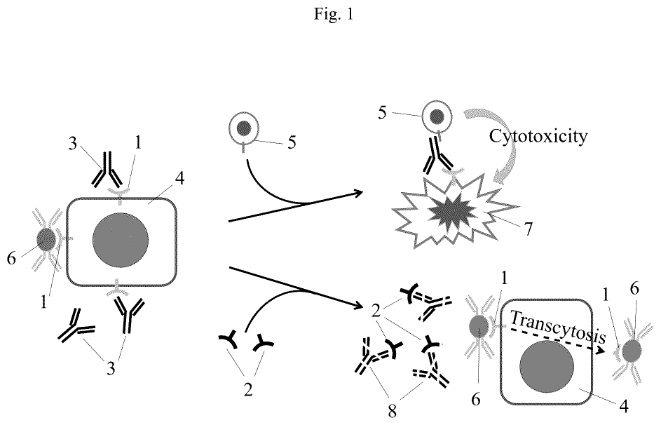

As is depicted in FIG. 1, under pathophysiological condition, autoantibodies recognize and bind endogenously expressed pIgR on the mucosal epithelia. This binding attracts immune cells and initiates autoimmune response, leading to mucosal epithelia injury. When pIgR mutant is administrated, it interacts with the autoantibodies. The neutralized autoantibodies then undergo proteolysis and degradation. Therefore, neutralization of the autoantibodies mitigates or eliminates autoimmunity and restores mucosal epithelia homeostasis and function.

The mutation may be insertion, deletion and/or replacement of one or more amino acid residues in the amino acid sequence of the extracellular portion of wild-type pIgR, and occur at one or more positions selected from a group consisting of positions 33-55, 114-117 and 486 in SEQ ID NO: 2.

In preferred embodiments, the mutation occurs at one or more positions selected from a group consisting of positions 48-50, 114-117 and 486 in SEQ ID NO: 2. By way of example, the pIgR mutant comprises one or more replacements of amino acid residues in SEQ ID NO: 2 selected from a group consisting of:
(i) ₄₈NRH to ₄₈TEL, or ₄₈NRH to ₄₈TAL;
(ii) ₁₁₄INSR to ₁₁₄ALSI, or ₁₁₄INSR to ₁₁₄ALST; and
(iii) C486S.

In order to facilitate the purification of the pIgR mutant or enhance its stability, the pIgR mutant may further comprise at least one polypeptide tag. For example, as a polypeptide tag, a human IgG1 Fc portion can be inserted at the carboxyl terminus of pIgR mutant, or a carrier protein such as albumin can be conjugated with the pIgR mutant at position 637 (Serine) of SEQ ID NO: 2.

Amino acids 1-18 of the extracellular portion of the wild-type human pIgR correspond to a signal peptide, which will be removed when the protein is secreted out of host cells. Therefore, in some embodiments, the amino acid sequence of the pIgR mutant comprises or consists of an amino acid sequence corresponding to positions 19-637 of any of SEQ ID NOs: 4-37.

In some embodiments, exemplary pIgR mutants are shown in any of SEQ ID NOs: 4-37.

In preferred embodiments, the amino acid sequence of the pIgR mutant comprises or consists of an amino acid sequence corresponding to positions 19-637 of any of SEQ ID NOs: 4-20, or has a sequence identity of at least 90%, such as 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% to an amino acid sequence corresponding to positions 19-637 of any of SEQ ID NOs: 4-20.

In preferred embodiments, the amino acid sequence of the pIgR mutant is shown in any of SEQ ID NOs: 4-20, or has a sequence identity of at least 90%, such as 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% to any of SEQ ID NOs: 4-20.

### Isolated Nucleic Acid

The present invention also provides an isolated nucleic acid encoding the amino acid sequence of the pIgR mutant described herein.

The nucleic acid molecules of the present disclosure can be generated from any source using sequence data accessible in the art and the sequence information provided herein. For example, the DNA sequence(s) coding for the pIgR mutant can be isolated independently from cells containing the mutant, cDNA and genomic libraries, viral genomes or any prior art vector known to include it. Alternatively, the nucleic acid molecules of the invention can also be generated by chemical synthesis in an automatized process (e.g. assembled from overlapping synthetic oligonucleotides or synthetic genes). Modification(s) can be generated by a number of ways known to those skilled in the art, such as chemical synthesis, site-directed mutagenesis, PCR mutagenesis, DNA shuffling, etc.

### Expression Vector

The present disclosure provides an expression vector comprising the isolated nucleic acid encoding the amino acid sequence described herein.

The expression vector in the present disclosure may be, for example, a DNA plasmid, a bacterial plasmid, a virus, etc. Non-limiting examples of expression vectors are described in, e.g. Paul et al., 2002, Nature Biotechnology, 19, 505; Miyagishi and Taira, 2002, Nature Biotechnology, 19, 497; Lee et al., 2002, Nature Biotechnology, 19, 500; and Novina et al. 2002, Nature Medicine, advance online publication doi: 10.1038/nm725. The expression vector may further contain a promoter operably linked to the encoding sequence of the amino acid sequence of the pIgR mutant, so that the promoter may initiate the expression of the encoding sequence after the expression vector enters the host cell. The expression vector can be introduced into the host cell by a suitable method such as, but not limited to, calcium phosphate transfection, lipofection transfection, electroporation transfection, bacterial heat shock, etc. For details, please refer to Sambrook et al. Molecular Cloning (a laboratory manual, Cold Spring Harbor, 1989).

### Host Cell

The present disclosure provides a host cell comprising the expression vector described herein. The host cell described in the present disclosure can be a eukaryotic cell or a prokaryotic cell. Suitable eukaryotic cells may include, for example, mammalian cells such as Chinese hamster ovary cells (CHO) or HEK293 cells, or insect cells such as Sf9 cells. Suitable prokaryotic cells may include, for example, bacteria such as E. coli.

### Method for Preparing the Mutant

The pIgR mutant provided herein can be prepared by techniques known in the art. For example, the pIgR mutant can be prepared by chemical synthesis or genetic engineering.

Chemical synthesis methods mainly include solid-phase synthesis and liquid-phase synthesis. Solid-phase polypeptide synthesis methods include, for example, the Merrifield solid-phase synthesis method, which is described in detail in the literature “Merrifield, J. Am. Chem. Soc. 85: 2149-2154” and “M. Bodanszky et al., Peptide Synthesis, John Wiley & Sons, Second Edition, 1976” and “J. Meienhofer, Hormonal Proteins and Peptides, Vol. 2, p. 46, Academic Press (New York), 1983”. The entire contents of these documents are hereby incorporated into this application as references. The Merrifield solid-phase synthesis mainly includes the following steps: attaching the protected C-terminal amino acid of the peptide to the resin based on the amino acid sequences of the target protein. After attachment, the resin is filtered, washed and the protecting group (e.g. t-butyloxycarbonyl) on the alpha amino group of the C-terminal amino acid is removed. The removal of this protecting group must take place, of course, without breaking the bond between that amino acid and the resin. The resulting resin peptide is then coupled to the penultimate C-terminal protected amino acid. This coupling takes place by the formation of an amide bond between the free carboxyl group of the second amino acid and the amino group of the first amino acid attached to the resin. This sequence of events is repeated with successive amino acids until all amino acids of the peptide are attached to the resin. Finally, the protected peptide is cleaved from the resin and the protecting groups removed to obtain the desired peptide.

The pIgR mutant disclosed herein can also be prepared by liquid-phase synthesis, for example, by the standard solution peptide synthesis, which has been described in “E. Schroder and K. Kubke, The Peptides, Vol. 1, Academic Press (New York), 1965” in detail, which is incorporated herein in its entirety by reference. Liquid-phase synthesis mainly includes coupling amino acids or peptide fragments step by step by chemical or enzymic methods that form amide bonds.

The genetic engineering method is a method of expressing a nucleic acid sequence encoding the corresponding pIgR mutant in a proper host cell to generate the corresponding mutant. For a detailed description of this method, see Sambrook et al. Molecular Cloning (a laboratory manual, Cold Spring Harbor, 1989). In some embodiments, the method for preparing the pIgR mutant provided herein comprises determining one or more positions for mutation, performing site-directed mutagenesis at said position on the full-length sequence of a plasmid comprising a nucleic acid sequence encoding the amino acid sequence of a wild-type pIgR, transfecting the plasmid with site-directed mutagenesis into a host cell, and inducing the host cell to produce the pIgR mutant.

The term “site-directed mutagenesis” refers to the introduction of an interested change into a target DNA fragment, including additions, deletions, and substitutions of bases, etc. In the present disclosure, the target DNA fragment is the encoding sequence of wild-type pIgR, e.g. Genbank NM_002644.4 (SEQ ID NO: 3). Accordingly, the mutated position can be located at positions 48-50, 114-117 and 486 in SEQ ID NO: 2, i.e., the amino acid sequence of the extracellular portion of wild-type human pIgR.

As an example, the site-directed mutagenesis comprises the following steps:
the nucleotide sequence of the entire coding region of the mutant is assembled from chemical synthetic oligo nucleotides where the mutation sites are embedded in the sequence and the codons are optimized for mammalian expression system.

As another example, the site-directed mutagenesis comprises the following steps:
two pairs of oligo nucleotides are synthesized as PCR primers. The first pair oligos contain a forward primer (F1) that completely matches the wild-type sequence and a reverse primer (MR1) containing the mutation site. The second pair oligos contain a forward primer (MF1) that bears the mutation site while a reverse primer (R2) completely matches the wild-type sequence. Of note, the primer MR1 and MF1 should share significant sequence overlap and complement to each other. Two rounds of PCR will be performed using high-fidelity Taq polymerase. The first round PCR generates two DNA fragments (i.e. 5′-portion and 3′-portion) using F1 and MR1, MF1 and R2, respectively. The second round PCR generates the target fragment using the above two fragments as template and F1 and R2 as primers. The target DNA fragment is then inserted into a plasmid vector using recombinant DNA techniques.

### Pharmaceutical Composition

The present disclosure provides a pharmaceutical composition comprising the pIgR mutant described herein and optionally a pharmaceutically acceptable carrier.

The term “pharmaceutically acceptable carrier” refers to a pharmaceutically acceptable solvent, suspending agent, or any other pharmacologically inert vehicles used to deliver the pIgR mutant to a subject without interfering the structure and properties of the pIgR mutant. Some of such carriers may enable the pIgR mutant to be formulated, for example, as tablets, pills, capsules, liquids, gels, syrups, slurries, suspensions and troches for oral administration to the subject. Some of such carriers enable the pIgR mutant to be formulated as injections, infusions or topical administration.

The pharmaceutically acceptable carriers for use in the pharmaceutical compositions of the present disclosure may include, but are not limited to, for example, pharmaceutically acceptable liquids, gels, or solid carriers, aqueous vehicles (e.g., sodium chloride injection, Ringer's injection, isotonic glucose injection, sterile water injection, or Ringer's injection of glucose and lactate), non-aqueous vehicles (e.g., fixed oils of vegetable origin, cottonseed oil, corn oil, sesame oil, or peanut oil), antimicrobial agents, isotonic agents (such as sodium chloride or dextrose), buffers (such as phosphate or citrate buffers), antioxidants (such as sodium bisulfate), anesthetics (such as procaine hydrochloride), suspending/dispending agents (such as sodium carboxymethylcellulose, hydroxypropyl methylcellulose, or polyvinylpyrrolidone), chelating agents (such as EDTA (ethylenediamine tetraacetic acid) or EGTA (ethylene glycol tetraacetic acid)), emulsifying agents (such as Polysorbate 80 (TWEEN-80)), diluents, adjuvants, excipients, or non-toxic auxiliary substances, other components known in the art, or various combinations thereof. Suitable components may include, for example, fillers, binders, disintegrants, buffers, preservatives, lubricants, flavorings, thickeners, coloring agents, or emulsifiers.

In some embodiments, the pharmaceutical composition is an oral formulation. The oral formulations include, but are not limited to, capsules, cachets, pills, tablets, troches (for taste substrates, usually sucrose and acacia or tragacanth), powders, granules, or aqueous or non-aqueous solutions or suspensions, or water-in-oil or oil-in-water emulsions, or elixirs or syrups, or confectionery lozenges (for inert bases, such as gelatin and glycerin, or sucrose or acacia) and/or mouthwash and its analogs.

In some embodiments, the oral solid formulation (e.g., capsules, tablets, pills, dragees, powders, granules, etc.) includes the pIgR mutant and one or more pharmaceutically acceptable carriers, such as sodium citrate or dicalcium phosphate, and/or the followings: (1) fillers or extenders such as starch, lactose, sucrose, glucose, mannitol and/or silicic acid; (2) binders such as, carboxymethylcellulose, alginates, gelatin, polyvinylpyrrolidone, sucrose, and/or acacia; (3) humectants such as glycerol; (4) cleaving agents such as agar, calcium carbonate, potato or tapioca starch, alginic acid, certain silicates, and sodium carbonate; (5) retarder solutions such as paraffin; (6) accelerating absorbers such as quaternary ammonium compounds; (7) lubricants such as acetyl alcohol and glycerol monostearate; (8) absorbents such as kaolin and bentonite; (9) lubricants such as talc, calcium stearate, magnesium stearate, solid polyethylene glycol, sodium sulfate, and mixtures thereof; and (10) colorants.

In some embodiments, the oral liquid formulation includes pharmaceutically acceptable emulsions, microemulsions, solutions, suspensions, syrups and elixirs, etc. In addition to the pIgR mutant, the liquid dosage forms may also contain conventional inert diluents such as water or other solvents, solubilizers and emulsifiers such as ethanol, isopropanol, ethyl carbonate, ethyl acetate, benzyl alcohol, benzene (meth) acrylate, propylene glycol, 1,3-butylene glycol, oils (in particular, cottonseed, groundnut, corn, olive, castor and sesame oils), glycerol, tetrahydrofurfuryl alcohol, polyethylene glycol and fatty acid sorbitol esters, and mixtures thereof. Besides inert diluents, the oral compositions may also contain adjuvants such as wetting agents, emulsifying and suspending agents, sweetening, flavoring, coloring, flavoring and preserving agents.

In some embodiments, the pharmaceutical composition may be an injectable formulation, including sterile aqueous solutions or dispersions, suspensions or emulsions. In all cases, the injectable formulation should be sterile and should be liquid to facilitate injections. It should be stable under the conditions of manufacture and storage, and should be resistant to the infection of microorganisms (such as bacteria and fungi). The carrier may be a solvent or dispersion medium containing, for example, water, ethanol, polyols (e.g., glycerol, propylene glycol, and liquid polyethylene glycols, etc.) and suitable mixtures and/or vegetable oils thereof. The injectable formulation should maintain proper fluidity, which may be maintained in a variety of ways, for example, using a coating such as lecithin, using a surfactant, etc. Antimicrobial contamination can be achieved by the addition of various antibacterial and antifungal agents (e.g., parabens, chlorobutanol, phenol, sorbic acid, thimerosal, etc.).

In some embodiments, the pharmaceutical composition is an oral spray formulation or nasal spray formulation. Such spray formulations include, but are not limited to, aqueous aerosols, non-aqueous suspensions, liposomal formulations, or solid particulate formulations, etc. Aqueous aerosols are formulated by combining an aqueous solution or suspension of the agent with a conventional pharmaceutically acceptable carrier and stabilizer. The carrier and stabilizer may vary according to the specific needs, but generally include nonionic surfactants (Tweens, or polyethylene glycol), oleic acid, lecithin, amino acids such as glycine, buffers, salts, sugar or sugar alcohol. Aerosols are usually prepared from isotonic solutions and can be delivered by nebulizers.

In some embodiments, the pharmaceutical compositions may be used in combination with one or more other drugs. In some embodiments, the composition comprises at least one other drug. In some embodiments, the other drug is one or more selected from a group consisting of ursodeoxycholic acid, fenofibrate, obeticholic acid, and the derivatives thereof.

In some embodiments, the pharmaceutical compositions may be delivered to the subject by suitable routes including, but not limited to, the oral route, injection route (e.g., intravenous injection, intramuscular injection, subcutaneous injection, intradermal injection, intracardiac injection, intrathecal injection, intrapleural injection, intraperitoneal injection, etc.), mucosal route (e.g., intranasal administration, oral administration, etc.), sublingual route, rectal route, transdermal route, intraocular route, pulmonary route. In some embodiments, the pharmaceutical compositions can be administered by injection route.

### Treatment of Autoimmune Diseases

As mentioned above, the pIgR mutant described herein can neutralize the autoantibodies against the polymeric immunoglobulin receptor to treat autoimmune diseases. Therefore, the present disclosure provides a method of treating, inhibiting, reducing the severity of, slowing progression of and/or promoting prophylaxis of autoimmune diseases in a subject in need thereof. The autoimmune diseases include but not limited to primary biliary cholangitis (PBC) and autoimmune hepatitis (AIH).

In some embodiments, the method comprises administering an effective amount of the pIgR mutant described herein or the pharmaceutical composition described herein to the subject in need thereof. In particular, the pIgR mutant is obtained in advance by the preparation method described above.

In some embodiments, the pIgR mutant is expressed in vivo in the subject in need thereof. Specifically, the method comprises the steps of administering an effective amount of the isolated nucleic acid described herein or the expression vector described herein to the subject; and expressing the pIgR mutant in vivo in the subject.

As an example, the expression vector can be a virus, such as adenovirus, an adenovirus-associated vector, or lentivirus.

As another example, the isolated nucleic acid can be the transcript mRNA of the pIgR mutant described herein. In other words, the transcript mRNA of the pIgR mutant can be administrated to the subject, for example, in a form of mRNA vaccine.

### EXAMPLES

The following examples are provided to better illustrate the claimed invention and are not to be interpreted as limiting the scope of the invention. To the extent that specific materials are mentioned, it is merely for purposes of illustration and not intended to limit the invention. One skilled in the art may develop equivalent means or reactants without the exercise of inventive capacity and without departing from the scope of the invention.

Seventeen human pIgR mutants, which are shown in Table 1, were prepared in Example 1, and among them, eight mutants were tested in Example 2 or 3.

TABLE 1

| Mutant | Mutation(s) | Sequence |
|---|---|---|
| 1 | ₄₈NRH to ₄₈TAL | SEQ ID NO: 5 |
| 2 | ₄₈NRH to ₄₈TEL | SEQ ID NO: 4 |
| 3 | ₁₁₄INSR to ₁₁₄ALSI | SEQ ID NO: 6 |
| 4 | ₁₁₄INSR to ₁₁₄ALST | SEQ ID NO: 7 |
| 5 | C486S | SEQ ID NO: 8 |
| 6 | ₄₈NRH to ₄₈TAL and ₁₁₄INSR to | SEQ ID NO: 12 |
| | ₁₁₄ALSI | |
| 7 | ₄₈NRH to ₄₈TAL and C486S | SEQ ID NO: 14 |
| 8 | ₄₈NRH to ₄₈TAL and C486S and | SEQ ID NO: 19 |
| | ₁₁₄INSR to ₁₁₄ALSI | |
| 9 | ₄₈NRH to ₄₈TEL and ₁₁₄INSR to | SEQ ID NO: 9 |
| | ₁₁₄ALSI | |
| 10 | ₄₈NRH to ₄₈TEL and ₁₁₄INSR to | SEQ ID NO: 10 |
| | ₁₁₄ALST | |
| 11 | ₄₈NRH to ₄₈TEL and C486S | SEQ ID NO: 11 |
| 12 | ₄₈NRH to ₄₈TAL and ₁₁₄INSR to | SEQ ID NO: 13 |
| | ₁₁₄ALST | |
| 13 | ₁₁₄INSR to ₁₁₄ALSI and C486S | SEQ ID NO: 15 |
| 14 | ₁₁₄INSR to ₁₁₄ALST and C486S | SEQ ID NO: 16 |
| 15 | ₄₈NRH to ₄₈TEL and ₁₁₄INSR to | SEQ ID NO: 17 |
| | ₁₁₄ALSI and C486S | |
| 16 | ₄₈NRH to ₄₈TEL and ₁₁₄INSR to | SEQ ID NO: 18 |
| | ₁₁₄ALST and C486S | |
| 17 | ₄₈NRH to ₄₈TAL and ₁₁₄INSR to | SEQ ID NO: 20 |
| | ₁₁₄ALST and C486S | |

### Example 1: Preparation of Human pIgR Mutants

I. Human pIgR Mutant with ₄₈NRH being Replaced with ₄₈TAL (Mutant 1)
(i) Cloning of Wild-Type Human pIgR Extracellular Portion

Two primers were made based on Genbank NM_002644.4 sequence (SEQ ID NO: 3) by adding a unique restriction endonuclease site at the 5′-end of each primer, respectively. The two primers were as follows:

pIgR-F1 (forward), inserted a Hind III site:

(SEQ ID NO: 38)
GCTACAAGCTTGCGGCGCTCGGGACCC

pIgR-R2 (reverse), inserted a EcoR I site:

(SEQ ID NO: 39)
CGATGAATTCAGCTTCCACCTTGTTCCTCAG

A high-fidelity Taq polymerase (Ref #04738292001, FastStart High Fidelity PCR System from Roche Diagnostics GmbH, Germany) was used to amplify the target DNA fragment from a cDNA library derived from human liver tissue. The amplified fragment was digested with Hind III and EcoR I (New England Biolab, MA) along with a plasmid vector pcDNA3.1. After DNA ligation, the recombinant construct was transformed into E. coli DH5a competent cells and plated on 1.5% agar plates containing LB-medium and 100 μg/ml ampicillin. After overnight incubation at 37° C., colonies were inoculated into 2-4 ml LB medium and grew at 37° ° C. for overnight. Plasmid DNA was extracted from the overnight culture using Qiagen Mini-prep Kit. The positive clones were identified by restriction digestion and further confirmed by DNA sequencing.

### (ii) Design of Mutagenesis Primers

The amino acids in human pIgR that directly interact with polymeric immunoglobins were selected for mutagenesis. Once the codons encoding these amino acids were located in the pIgR cDNA sequence, they would be replaced with codons that encode mutant amino acids. A set of two mutagenesis primers (one forward, and another reverse) were designed that contain this sequence with additional 6-10 base pair (bp) upstream and 15-24 bp downstream of the mutation site identical to those in the wild-type cDNA. In this example, ₄₈NRH were changed to ₄₈TAL in human pIgR, two mutagenesis primers were made as following:

MF1A forward primer:

(SEQ ID NO: 40)
GTCACCGCGCTCACCCGGAAGTACTGGTGC

MR1A reverse primer:

(SEQ ID NO: 41)
GGGTGAGCGCGGTGACAGAGGTGGGTGGGTAG

(iii) PCR Procedure

To create the ₄₈NRH to ₄₈TAL mutation, two rounds of PCR were performed. In the first round, primers pIgR-F1 and MR1A were used to amplify the 5′-portion, and primers MF1A and pIgR-R2 were used to amply the 3′-portion from wild-type pIgR cDNA expression construct. Specifically, the condition was shown in below:

| Cycle step | Temperature | Time | | Cycle number |
|---|---|---|---|---|
| Initial denaturation | 94° C. | 3 | min | 1 |
| Denaturation | 94° C. | 30 | sec | 25 |
| Annealing | 58° C. | 30 | sec | |
| Extension | 72° C. | 1.5 | min | |
| Final extension | 72° C. | 10 | min | 1 |

The desired DNA fragments were recovered after agarose gel electrophoresis.

In the second round, the first round PCR product that bears the mutation sites was used as the template, and pIgR-F1 and pIgR-R2 were used as the primers with similar PCR condition except that the extension step lasted for 2 min in each cycle. This generated a DNA fragment that encodes the extracellular portion of human pIgR with ₄₈NRH mutated to ₄₈TAL. This DNA was inserted into pcDNA3.1 vector as mentioned earlier.

(iv) Expression of the pIgR Mutant

HEK293 cells and CHO cells were used to express the recombinant proteins of the extracellular portion of human pIgR wild-type or mutants. The cells were maintained in a 37° C., 5% CO₂ atmosphere incubator. Specifically, plasmid constructs were transiently transfected into these cells when they reached to ˜75% confluence using Lipofectamine 2000 (ThermoFisher Scientific). Typically, for each well in a 6-well plate, 2-2.5 μg of DNA and 10 μl Lipofectamine 2000 were used in the transfection. 16 hours after the transfection, the culture medium was replaced with 2% FBS-DMEM. 40 hours after the transfection, the culture medium was collected and replaced with fresh 2% FBS-DMEM. 64 hours after the transfection, both the culture medium and cells were collected. The culture media collected at 40 hours and 64 hours after the transfection were combined. The recombinant proteins in the culture media and cell lysates were assayed for molecular weight, and binding activity for polymeric immunoglobulins. To enrich/purify the 6×His tagged recombinant proteins, Ni-NTA beads/column from Qiagen were used.

II. Human pIgR Mutant with a Polypeptide Tag

To tag the pIgR mutants with 6×His tag, a PCR procedure was performed as mentioned above, except that the reverse primer pIgR-R2 was replaced with primer pIgR-R3, the nucleotide sequence of which is:

(SEQ ID NO: 42)
CGATGAATTCAGTGATGGTGATGGTGATGGGAGCTTCCACCTTGTTCCTC

To tag the pIgR mutants with a human IgG1 Fc portion, two rounds of PCR that were similar to creating mutations described above were performed, except that the primer sets were pIgR-F1 with pIgR-IgG1-R (for first round PCR to obtain the 5′ portion), and pIgR-IgG1-F with IgG1Fc-R2 (for first round PCR to obtain the 3′ portion). To obtain the full-length of the fusion protein, pIgR-F1 and IgG1Fc-R2 were used as a primer set for the second round PCR.

pIgR-IgG1-F:

(SEQ ID NO: 43)
TCTGAGGAACAAGGTGGAAGCGACAAAACTCACACATGCCCAC

pIgR-IgG1-R:

(SEQ ID NO: 44)
GTGGGCATGTGTGAGTTTTGTCGCTTCCACCTTGTTCCTCAGA

### IgG1Fc-R2:

(SEQ ID NO: 45)
CGATGAATTCGCACTCATTTACCCGGAG

III. Other Human pIgR Mutants

Other human pIgR mutants were also prepared, including the pIgR mutant with ₄₈NRH being replaced with ₄₈TEL (Mutant 2), the pIgR mutant with ₁₁₄INSR being replaced with ₁₁₄ALSI (Mutant 3), the pIgR mutant with ₁₁₄INSR being replaced with ₁₁₄ALST (Mutant 4), and the pIgR mutant with a mutation of C486S (Mutant 5), the pIgR mutant with ₄₈NRH being replaced with TAL and ₁₁₄INSR being replaced with ALSI (Mutant 6), the pIgR mutant with ₄₈NRH being replaced with TAL and a mutation of C486S (Mutant 7), and the pIgR mutant with ₄₈NRH being replaced with TAL and a mutation of C486S and ₁₁₄INSR being replaced with ALSI (Mutant 8).

The preparation processes of Mutants 2-5 were similar to that of Mutant 1 except for using the primer sets listed in Table 2 as the mutagenesis primers.

TABLE 2

| Mutant | Primer | Sequence |
|---|---|---|
| 2 | MF1E forward | GTCACCGAGCTCACCCGGAAGTACTGG |
| | primer | TGC (SEQ ID NO: 46) |
| | MR1E reverse | GGGTGAGCTCGGTGACAGAGGTGGGTG |
| | primer | GGTAG (SEQ ID NO: 47) |
| 3 | MINSR2I forward | GGCGCCCTTAGCATAGGCCTGTCCTTT |
| | primer | GATGTCA (SEQ ID NO: 48) |
| | MINSR2I reverse | GCCTATGCTAAGGGCGCCCAGGCCACA |
| | primer | CTTGT (SEQ ID NO: 49) |
| 4 | MINSR2T forward | GGCGCCCTTAGCACAGGCCTGTCCTTT |
| | primer | GATGTCAG (SEQ ID NO: 50) |
| | MINSR2T reverse | GCCTGTGCTAAGGGCGCCCAGGCCACA |
| | primer | CTTGT (SEQ ID NO: 51) |
| 5 | MC2S forward | CCAAGCAAATTCTCCTCGTACGAGAAA |
| | primer | TACT (SEQ ID NO: 52) |
| | MC2S reverse | CGTACGAGGAGAATTTGCTTGGAAAGT |
| | primer | GACAGGGGACC (SEQ ID NO: 53) |

To generate Mutant 6 and Mutant 7, Mutant 1 was used as the template DNA for PCRs. For creating Mutant 8, Mutant 7 was used as the template DNA for PCRs. Mutants 9-17 were also prepared using similar methods.

### Example 2: Human pIgR Mutants do not Interact with Human dIgA and pIgM

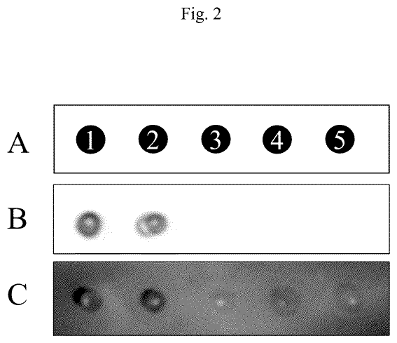

HEK293 cells were transiently transfected with the wild-type and the mutant expression constructs. Both the culture medium and cell pellet were collected 48-72 hr post transfection. The cell pellet was lysed with RIPA or a phosphate buffer, and the supernatant was collected after 10K×g centrifugation for 10 min. To purify/enrich the exogenously expressed polypeptide-tagged recombinant proteins in the culture media and cell lysate, Ni-NTA beads or Protein A/G beads were used for 6×His or human IgG1 Fc portion tagged proteins, respectively. 0.5 M imidazole or 0.1 M citrate (pH2.5) was used to elute/recover the recombinant proteins tagged with 6×His or IgG1 Fc portion, respectively. To characterize the biological activities of the recombinant proteins, 0.5-1 nanogram (in 1-2 microliter) of each purified recombinant protein was spotted on a nitrocellulose paper, the dot blot was blocked with 4% non-fat milk. The blot was then incubated with horseradish peroxidase conjugated human dIgA (dIgA-HRP) or pIgM-HRP (each ˜0.5 microgram/ml in 0.1% BSA) at room-temperature for 1 hr. After the blot was washed for three times, the blot was developed with ECL reagent. The result was shown in FIG. 2.

FIG. 2 demonstrates that the wild-type pIgR and the V28A mutant (SEQ ID NO: 54) bind well with human dIgA and pIgM. In contrast, the “NRH to TAL” mutant (Mutant 1), “NRH to TEL” mutant (Mutant 2) and the “NRH to TAL and INSR to ALSI” mutant (Mutant 6) bind neither dIgA nor pIgM, indicating that these mutation sites disable pIgR interaction with polymeric immunoglobulins in the blood.

### Example 3: Human pIgR Mutants can Neutralize Autoantibodies from the Serum of PBC Patients

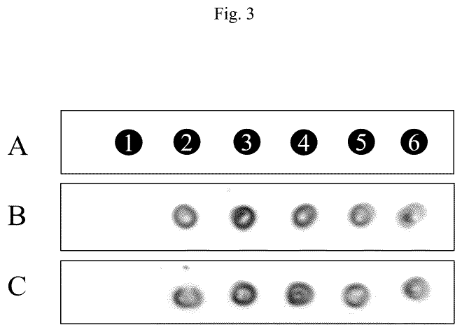

To test whether the recombinant pIgR proteins interact with autoantibody IgG found in PBC patients, dot blot was performed in which the exogenously expressed pIgR mutants were incubated with serum (1:10 to 1:100 diluted in PBS-T) from a PBC patient or with a commercial polyclonal rabbit antibody (ThermoFisher Scientific Inc.) against human pIgR (as positive control, 1:500 dilution) at room temperature for 1 hr, followed by incubation with HRP conjugated anti-human IgG or anti-rabbit IgG as a secondary antibody (1:5000 dilution). The dot blot was developed with ECL reagent. The result was shown in FIG. 3.

FIG. 3 shows that both the wild-type and the mutants interact with the autoantibodies in PBC patients.

The results as shown in FIG. 2 and FIG. 3 indicate that the exogenously expressed human pIgR mutants described herein are able to neutralize the autoantibodies from the patient, but not interfere the normal physiologic functions of endogenous pIgR (binds dIgA and pIgM and facilitates their transcytosis) in the body.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present disclosure. Still further variations and alternate elements will be apparent to one of skill in the art. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about”. Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is to be understood that the embodiments of the invention disclosed herein are illustrative of the principles of the present invention. Other modifications that can be employed can be within the scope of the invention. Thus, by way of example, but not of limitation, alternative configurations of the invention can be utilized in accordance with the teachings herein. Accordingly, embodiments of the present disclosure are not limited to that precisely as shown and described.

1. A polymeric immunoglobulin receptor (pIgR) mutant for neutralizing an autoantibody against pIgR without interacting with a polymeric immunoglobulin, comprising an extracellular portion of wild-type pIgR with at least one amino acid mutation therein.

2. The pIgR mutant of claim 1, wherein the mutation is insertion, deletion and/or replacement of one or more amino acid residues in the amino acid sequence of the extracellular portion of wild-type pIgR.

3. The pIgR mutant of claim 2, wherein the extracellular portion of wild-type pIgR is the extracellular portion of wild-type human pIgR.

4. The pIgR mutant of claim 3, wherein the amino acid sequence of the extracellular portion of wild-type human pIgR is shown in SEQ ID NO: 2.

5. The pIgR mutant of claim 4, wherein the mutation occurs at one or more positions selected from a group consisting of positions 33-55, 114-117 and 486 in SEQ ID NO: 2.

6. The pIgR mutant of claim 5, wherein the mutation occurs at one or more positions selected from a group consisting of positions 48-50, 114-117 and 486 in SEQ ID NO: 2.

7. The pIgR mutant of claim 6, wherein the pIgR mutant comprises one or more replacements of amino acid residues in SEQ ID NO: 2 selected from a group consisting of:
(i) ₄₈NRH to ₄₈TEL, or ₄₈NRH to ₄₈TAL;
(ii) ₁₁₄INSR to ₁₁₄ALSI, or ₁₁₄INSR to ₁₁₄ALST; and
(iii) C486S.

8. The pIgR mutant of claim 7, wherein the pIgR mutant further comprises at least one polypeptide tag.

9. The pIgR mutant of claim 8, wherein the polypeptide tag is selected from a group consisting of a 6×His tag, a human IgG Fc portion, and a carrier protein.

10. The pIgR mutant of claim 9, wherein the 6×His tag or the human IgG Fc portion is inserted at the carboxyl terminus of the pIgR mutant.

11. The pIgR mutant of claim 9, wherein, the carrier protein is conjugated with the pIgR mutant at position 637 in SEQ ID NO: 2.

12. The pIgR mutant of claim 7, wherein the amino acid sequence of the pIgR mutant comprises or consists of an amino acid sequence corresponding to positions 19-637 of any of SEQ ID NOs: 4-37.

13. The pIgR mutant of claim 12, wherein the amino acid sequence of the pIgR mutant is shown in any of SEQ ID NOs: 4-37.

14. The pIgR mutant of claim 7, wherein the amino acid sequence of the pIgR mutant comprises or consists of an amino acid sequence corresponding to positions 19-637 of any of SEQ ID NOs: 4-20, or has a sequence identity of 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% to an amino acid sequence corresponding to positions 19-637 of any of SEQ ID NOs: 4-20.

15. The pIgR mutant of claim 14, wherein the amino acid sequence of the pIgR mutant is shown in any of SEQ ID NOs: 4-20, or has a sequence identity of 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% to any of SEQ ID NOs: 4-20.

16. The pIgR mutant of claim 1, wherein the pIgR mutant does not interact with dIgA and/or pIgM.

17. An isolated nucleic acid encoding the amino acid sequence of the pIgR mutant of any one of claims 1-16.

18. A pharmaceutical composition comprising the pIgR mutant of any one of claims 1-16, and optionally a pharmaceutically acceptable excipient, carrier or vehicle.

19. A method of treating, inhibiting, reducing the severity of, slowing progression of and/or promoting prophylaxis of an autoimmune disease in a subject in need thereof comprising:
administering to the subject an effective amount of the pIgR mutant of any one of claims 1-16 or the pharmaceutical composition of claim 18;
or
administering to the subject an effective amount of the isolated nucleic acid of claim 17 or an expression vector comprising the same, and
expressing the pIgR mutant in vivo in the subject.

20. The method of claim 19, wherein the autoimmune disease is selected from a group of consisting of primary biliary cholangitis and autoimmune hepatitis.


SEQUENCE LISTING (54 sequences)

SEQ ID NO: 1 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 2 - protein, 637 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 3 - RNA, 4275 nt, Homo sapiens
  source 1..4275: mol_type = mRNA
gagcagagtttcagttttggcagcagcgtccagtgccctgccagtagctcctagagaggcaggggttaccaactggccagcaggctgtgtccctgaagtcagatcaacgggagagaaggaagtggctaaaacattgcacaggagaagtcggcctgagtggtgcggcgctcgggacccaccagcaatgctgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagagcgctggtctccaccctggtgcccctgggcctggtgctggcagtgggagccgtggctgtgggggtggccagagcccggcacaggaagaacgtcgaccgagtttcaatcagaagctacaggacagacattagcatgtcagacttcgagaactccagggaatttggagccaatgacaacatgggagcctcttcgatcactcaggagacatccctcggaggaaaagaagagtttgttgccaccactgagagcaccacagagaccaaagaacccaagaaggcaaaaaggtcatccaaggaggaagccgagatggcctacaaagacttcctgctccagtccagcaccgtggccgccgaggcccaggacggcccccaggaagcctagacggtgtcgccgcctgctccctgcacccatgacaatcaccttcagaatcatgtcgatcctggggccctcagctcctggggaccccactccctgctctaacacctgcctaggtttttcctactgtcctcagaggcgtgctggtcccctcctcagtgacatcaaagcctggcctaattgttcctattggggatgagggtggcatgaggaggtcccacttgcaacttctttctgttgagagaacctcaggtacggagaagaatagaggtcctcatgggtcccttgaaggaagagggaccagggtgggagagctgattgcagaaaggagagacgtgcagcgcccctctgcacccttatcatgggatgtcaacagaatttttccctccactccatccctccctcccgtccttcccctcttcttctttccttccatcaaaagatgtatttgaattcatactagaattcaggtgctttgctagatgctgtgacaggtatgccaccaacactgctcacagcctttctgaggacaccagtgaaagaagccacagctcttcttggcgtatttatactcactgagtcttaacttttcaccaggggtgctcacctctgcccctattgggagaggtcataaaatgtctcgagtcctaaggccttaggggtcatgtatgatgagcatacacacaggtaattataaacccacattcttaccatttcacacataagaaaattgaggtttggaagagtgaagcgtttttctttttcttttttttttttgagacggagtctctcactgtcgcccaggctggagtgcagtggcgcaatctcggctcactgcaacctccgcctcccaggttgacaccattctcctgcctcaccctcccaagtagctgggactacaggcgcctgccagcacgcctggctaattttttgtatttttagtagagacagggtttcaccgtgttagccaggatggtctcgatctcctgacctcgtgatccgcctgcctctgcctcccaaagtgctgggattacaggcgtgagccaccgcgtccggcctctttttttcttttcttttttttgagacaaagtctcactgtgtcacccagactggaatgcagtgacacaatctcggctcactgaaacctctgccttccaggttcaagctattctcatgcctcagcctctcaagtagctgggactacagatgtgggccaccatgtctggctaattttttttttttttttttttttttgtagagacagggtttcgccatgttgacgagactggtctcgaactcctggcctcaagtgatctgccgcctcagcttctcaaagtactgggattatataggcatgagccactgagcctggccctgaagcgtttttctcaaaggccctcagtgagataaattagatttggcatctcctgtcctgggccagggatctctctacaagagcccctgcccctctgttggaggcacagttttagaataaggaggaggagggagaagagaaaatgtaaaggagggagatctttcccaggccgcaccatttctgtcactcacatggacccaagataaaagaatggccaaaccctcacaacccctgatgtttgaagagttccaagttgaagggaaacaaagaagtgtttgatggtgccagagaggggctgctctccagaaagctaaaatttaatttcttttttcctctgagttctgtacttcaaccagcctacaagctggcacttgctaacaaatcagaaatatgacaattaatgattaaagactgtgattgccaccaa

SEQ ID NO: 4 - protein, 637 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 5 - protein, 637 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 6 - protein, 637 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 7 - protein, 637 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 8 - protein, 637 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 9 - protein, 637 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 10 - protein, 637 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 11 - protein, 637 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 12 - protein, 637 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 13 - protein, 637 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 14 - protein, 637 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 15 - protein, 637 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 16 - protein, 637 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 17 - protein, 637 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 18 - protein, 637 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 19 - protein, 637 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 20 - protein, 637 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS

SEQ ID NO: 21 - protein, 764 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 22 - protein, 764 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 23 - protein, 764 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 24 - protein, 764 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 25 - protein, 764 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 26 - protein, 764 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 27 - protein, 764 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 28 - protein, 764 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 29 - protein, 764 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 30 - protein, 764 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 31 - protein, 764 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 32 - protein, 764 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 33 - protein, 764 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 34 - protein, 764 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 35 - protein, 764 aa, synthetic construct
MLLFVLTCLLAVFPAISTKSPIFGPEEVNSVEGNSVSITCYYPPTSVTELTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 36 - protein, 764 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGALSIGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 37 - protein, 764 aa, synthetic construct
MLLFVLTCLLAVFPAISTKSPIFGPEEVNSVEGNSVSITCYYPPTSVTALTRKYWCRQGARGGCITLISSEGYVSSKYAGRANLTNFPENGTFVVNIAQLSQDDSGRYKCGLGALSTGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPSKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 38 - DNA, 27 bp, synthetic construct
  source 1..27: mol_type = other DNA
gctacaagcttgcggcgctcgggaccc

SEQ ID NO: 39 - DNA, 31 bp, synthetic construct
  source 1..31: mol_type = other DNA
cgatgaattcagcttccaccttgttcctcag

SEQ ID NO: 40 - DNA, 30 bp, synthetic construct
  source 1..30: mol_type = other DNA
gtcaccgcgctcacccggaagtactggtgc

SEQ ID NO: 41 - DNA, 32 bp, synthetic construct
  source 1..32: mol_type = other DNA
gggtgagcgcggtgacagaggtgggtgggtag

SEQ ID NO: 42 - DNA, 50 bp, synthetic construct
  source 1..50: mol_type = other DNA
cgatgaattcagtgatggtgatggtgatgggagcttccaccttgttcctc

SEQ ID NO: 43 - DNA, 43 bp, synthetic construct
  source 1..43: mol_type = other DNA
tctgaggaacaaggtggaagcgacaaaactcacacatgcccac

SEQ ID NO: 44 - DNA, 43 bp, synthetic construct
  source 1..43: mol_type = other DNA
gtgggcatgtgtgagttttgtcgcttccaccttgttcctcaga

SEQ ID NO: 45 - DNA, 28 bp, synthetic construct
  source 1..28: mol_type = other DNA
cgatgaattcgcactcatttacccggag

SEQ ID NO: 46 - DNA, 30 bp, synthetic construct
  source 1..30: mol_type = other DNA
gtcaccgagctcacccggaagtactggtgc

SEQ ID NO: 47 - DNA, 32 bp, synthetic construct
  source 1..32: mol_type = other DNA
gggtgagctcggtgacagaggtgggtgggtag

SEQ ID NO: 48 - DNA, 34 bp, synthetic construct
  source 1..34: mol_type = other DNA
ggcgcccttagcataggcctgtcctttgatgtca

SEQ ID NO: 49 - DNA, 32 bp, synthetic construct
  source 1..32: mol_type = other DNA
gcctatgctaagggcgcccaggccacacttgt

SEQ ID NO: 50 - DNA, 35 bp, synthetic construct
  source 1..35: mol_type = other DNA
ggcgcccttagcacaggcctgtcctttgatgtcag

SEQ ID NO: 51 - DNA, 32 bp, synthetic construct
  source 1..32: mol_type = other DNA
gcctgtgctaagggcgcccaggccacacttgt

SEQ ID NO: 52 - DNA, 31 bp, synthetic construct
  source 1..31: mol_type = other DNA
ccaagcaaattctcctcgtacgagaaatact

SEQ ID NO: 53 - DNA, 38 bp, synthetic construct
  source 1..38: mol_type = other DNA
cgtacgaggagaatttgcttggaaagtgacaggggacc

SEQ ID NO: 54 - protein, 637 aa, synthetic construct
MLLFVLTCLLAVFPAISTKSPIFGPEEANSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSS